(19) United States
(12) Patent Application Publication
(10) Pub. No.: US 2004/0092813 A1
(43) Pub. Date: May 13, 2004

(54) Magnetic resonance imaging apparatus

(76) Inventors: Masahiro Takizawa, Chiba (JP); Tetsuhiko Takahashi, Saitama (JP); Hisako Nagao, Chiba (JP); Yumiko Yatsui, Chiba (JP); Hidekazu Nakamoto, Chiba (JP)

Correspondence Address: Ivan S Kavrukov, Cooper & Dunham, 1185 Avenue of the Americas, New York, NY, 10036, US

(21) Appl. No.: 10/469,566

(22) PCT Filed: Feb. 28, 2002
(86) PCT No.: PCT/JP02/01851

(30) Foreign Application Priority Data
Mar. 1, 2001 (JP) 2001-57274
Jun. 15, 2001 (JP) 2001-732623

(51) Int. Cl. A61B 5/055

(52) U.S. Cl. 600/423

(57) ABSTRACT

An imaging sequence for MRI is performed to produce MR images of an object to be examined and display them on a monitor. Marks 6, 7 and 8 are defined at regions to be observed (observation regions), such as a bifurcation 3 and constriction 4 of the displayed image 1. If the distance between an invasive device 10 displayed in the image and the marks is within a predetermined range, at least one of the frame rate (reciprocal of an image-updating interval) and the spatial resolution is altered so as to automatically raise the imaging speed or so as to improve the spatial resolution when the invasive device reaches the observation region. The movement of the invasive device and its relationship to a blood vessel can therefore be finely monitored with high accuracy. When the invasive device is tracked, at least two distinctive points displayed in the MR image are detected and the three-dimensional position and moving direction of the invasive device are detected from angles between the line connecting these two points and three orthogonal axes.

### FIELD OF INVENTION

The present invention relates to a magnetic resonance imaging apparatus (abbreviated to MRI apparatus hereinafter). In particular, it relates to a technique for monitoring an invasive device such as a catheter inserted into the body of a patient, i.e., an object to be examined, while performing continuous imaging.

### RELATED ART

The MRI apparatus is an apparatus for imaging a region within an object (living body) based on magnetic resonance information such as the density distribution or relaxation time of hydrogen or phosphor determined by applying RF magnetic field pulses to the object placed in a uniform static magnetic field to excite the atomic nuclei of hydrogen or phosphor and measuring nuclear magnetic resonance signals (NMR signals) generated from the excited atomic nuclei and thereby facilitating diagnosis.

Medical applications of such an MRI apparatus include interventional MRI (abbreviated to I-MRI hereinafter). There has long been known an interventional technique of performing examination or treatment under X-ray fluoroscopy using an X-ray imaging apparatus. However, as a result of advances in the development of open-type MRI apparatuses that maximize the open space surrounding the imaging region of the patient so as to facilitate access to the patient by the operator, examinations or treatments under MRI fluoroscopy i.e., I-MRI, have been applied clinically. Examples of clinical I-MRI applications include biopsy using a biopsy needle, laser therapies, operations using a catheter and so forth.

Such I-MRI enables a device such as catheter that is inserted into an object (hereinafter referred as an invasive device) to be monitored and guided to a targeted region. Tracking techniques for detecting the position of the invasive device in real time have been developed for monitoring the invasive device. One of such techniques is known as the passive tracking method. In this method, a magnetic material included in resin constituting the tip of the invasive device distorts the static magnetic field in the vicinity of the tip of the invasive device, so that NMR signals disappear in the vicinity of the tip to make the MR image of the tip distinguishable.

Another technique is known as an active tracking method. In this method, the catheter or other suchinvasive device is equipped with a coil at the tip, which also receives NMR signals for producing an image. The image is superimposed on images of NMR signals received by the ordinary receiving coil and displayed on a monitor so that the tip of the invasive device is displayed at a high luminance.

Further, in “Active MR Guidance of Interventional Devices with Target-Navigation” Magnetic Resonance in Medicine, 44, pp.56-65 (2000), for example, there is set out another method in which a plurality of RF coils are embedded in a catheter to be spaced in the longitudinal direction of the catheter, and the direction of movement of the catheter is detected based on MR images of these coils. This enables an imaging slice (scan slice) including the path of the catheter and the location of diseased tissue of interest to be determined automatically. Such ability to automatically determine a scan slice and introduce a catheter to a region of interest based on positional information on the catneter is referred to as navigation capability. According to this method, the three-dimensional position and direction of the catheter can also be detected and the imaging slice including the tissue of interest and the catheter can be determined constantly without losing sight of the catheter.

On the other hand, I-MRI employs fast imaging fluoroscopy in order to monitor the position of the invasive device or the state of the patient in real time. The fluoroscopy performs an imaging sequence with a repetition time of several milliseconds (ms) to obtain images with an image-updating interval of about one second (s) or less. One fluoroscopic technique proposed for reducing image acquisition time is the echo-sharing method in which MR measurement is performed only partially and an image is produced by making up for the image data deficiency with previously obtained image data. The image-updating interval can be shortened to several tens of milliseconds by this technique.

While employment of an invasive device tracking technique or a continuous imaging method such as fluoroscopy enables real-time monitoring of the invasive device in I-MRI, conventional invasive device monitoring still has room for improvement in some points such as the spatial or temporal resolution of the obtained images, instantaneity (real-time property), artifacts etc., as explained hereinafter.

One of these is the problem that while the image spatial resolution or temporal resolution should change as the invasive device advances, conventional I-MRI fluoroscopy cannot satisfy this requirement. For instance, although insertion of the invasive device into a living body should always be done carefully, particular care may be required depending on the site of the living body. Specifically, since the internal structure of the patient differes locally, special carefulness is required when the device goes through, for example, a bifurcation, flection or constriction of a blood vessel or the device reaches an operated site. In such a site or region, it is required to shorten the image-updating interval or improve the spatial resolution in order to image the invasive device precisely.

In conventional I-MRI fluoroscopy, however, the image-updating interval and the spatial resolution are determined at the time the imaging sequence is determined. Therefore, depending on the site of the living body where the invasive devise is present, the invasive device cannot be imaged precisely because of the long image-updating interval or low spatial resolution, which affects the careful operation of the invasive device.

Another problem is that the instantaneity (real-time property) of the active tracking is degraded when the position and direction of the invasive device are detected three-dimensionally. In the conventional technique, it is necessary to measure three orthogonal slices twice in order to detect the three-dimensional position and direction of the invasive device, and thereafter the imaging slice including the target issue and the invasive device is determined to perform the actual scan imaging. Accordingly, detection of the catheter position has a time lag and navigation cannot be done in real time.

Therefore, an object of the present invention is, in performing I-MRI in which an invasive device inserted into an object to be examined is monitored, to image the invasive device and the site or region where the device is moving with a desired resolution. That is, an object of the present invention is to improve imaging of an invasive devise in a monitored image as the invasive device proceeds, and thereby improving the operability of the invasive device. Another object of the present invention is to track an invasive device with a short image-updating interval without losing sight of the invasive device during the tracking procedure. Yet another object of the present invention is to improve instantaneity of real-time guidance when the invasive device is guided to a region of interest while sequentially using the tracking result to automatically change the scan.

### SUMMARY OF INVENTION

In order to attain the above-mentioned objects, one aspect of an MRI apparatus of the present invention is characterized in that it is made capable of changing an imaging sequence based on the position information of the invasive device (including a distance from the region of interest and speed information) so that spatial resolution or temporal resolution of monitoring images is altered. Another aspect of the MRI apparatus of the present invention is characterized in that it employs an invasive device having two or more distinctive points along with the longitudinal direction and is provided with tracking capability for detecting the three-dimensional position and direction of movement of the invasive device based on three-dimensional images acquired continuously. The MRI apparatus may be provided with these features individually or in combination.

Specifically, the MRI apparatus of the present invention includes control means for performing an imaging sequence of measuring NMR signals generated by exciting an object to be examined and imparted with spatial position information, image construction means for producing magnetic resonance images (MR images) of the object based on the NMR signals, display means for displaying the images produced by the image construction means, input means for defining at least one mark at a desired position on the image displayed on the display means, wherein the control means is capable of altering the imaging sequence when the distance between the invasive device and the mark displayed in the image becomes within a predetermined range.

Alteration of the imaging sequence preferably involves alteration of at least one of the frame rate (reciprocal of image-updating interval) and the spatial resolution to, for example, a higher value.

The aforementioned problem can be solved by the invention as explained next. First, upon inserting an invasive device into a living body, the operator watches the MR images displayed on the display means to discern any bifurcation, constriction or the like of a blood vessel or the like where insertion of the invasive device should be conducted carefully, and defines a mark at the region (observation region) through the input means. If a surgical operation site is present, the operation site is also marked as an observation region. Then the control means tracks the position of the invasive device displayed in the image and alters the imaging sequence when the invasive device advances to within the marked range so that at least one of the frame rate and spatial resolution of image becomes a higher value. As a result, the imaging speed becomes faster or the spatial resolution becomes higher automatically when the invasive device arrives at the observation region, and the operator can finely monitor the movement of the invasive device and the precise positional relation to the blood vessel from the image.

In order to alter the frame rate and spatial resolution, a plurality of imaging sequences having different frame rates and spatial resolutions may be set previously and switched by the control means. Alternatively, the imaging sequence may be altered by changing parameters of the imaging sequence relating to the frame rate and spatial resolution. For example, the spatial resolution can be improved by making the field of view smaller.

Whether the position of the invasive device is within the marked range or not may be detected by a tracking means as follows. Specifically, the tracking means detects the invasive device in images based on difference of luminance or the like. And change of a position of the invasive device is detected every time when the image is updated to track the position. On the other hand, the position of a mark defined in the image is found and the distance between the mark and the invasive device is determined by operation. When the thus determined distance is within the predetermined range, it is judged that the invasive device is within the observation region and the frame rate of imaging or the spatial resolution is altered to a higher value so as to improve visibility of the movement of the invasive device or ability to image the invasive device. Both of the frame rate and the spatial resolution may be increased.

Thanks to these features, when the invasive device arrives at the observation region where the device must be treated carefully, the frame rate is shortened or the spatial resolution is improved automatically and the movement of the invasive device can be finely tracked. As a result, insertion of the invasive device can be done easily.

Instead of defining marks at the image observation regions, the moving speed of the invasive device may be found based on the positional change of the invasive device detected by the tracking means. In this case, if the moving speed of the invasive device is less than a predetermined value, for example, at least one of the frame rate and spatial resolution of image is altered to a higher value. That is, the operator generally becomes more careful when the invasive device arrives at an observation position such as a bifurcation of the blood vessel, so that the insertion speed becomes slow naturally. This lowered speed is utilized for altering the frame rate and spatial resolution. The same effect can be obtained as in the case of defining a mark.

Further, the MRI apparatus according to the present invention includes control means for performing imaging sequence of measuring NMR signals generated by exciting an object to be examined and imparted with spatial position information, and image construction means for continuously producing magnetic resonance images (MR images) of the object based on the NMR signals, wherein the control means includes invasive device detecting means for detecting the three-dimensional position and direction of the invasive device based on images of at least two distinctive points incorporated in the invasive device.

Specifically, in this MRI apparatus, on condition that imaging is performed using an invasive device provided with at least two distinctive points spaced in the longitudinal direction, the invasive device detecting means detects the distinctive points of the invasive device inserted into the object based on MR images and detects the position and three-dimensional direction of the invasive device by determining the direction of a line between the two distinctive points. Each distinctive point is a point distinguishable from the other portions of the MR image.

According to the present invention, the distinctive point may be formed, for example, by embedding a small RF receiving coil in the tip of the invasive device or by admixing a marker having a high signal intensity material or low signal intensity material such as a magnetic material with a resin of a catheter.

A two-dimensional or three-dimensional imaging sequence may be employed for the imaging sequence of the invention. When a two-dimensional imaging sequence is employed, three orthogonal axis sections are imaged and a straight line connecting the distinctive points is found from the three-axis section images. The three-axis section images include, for example, a section in the coronal direction of a patient lying on his or her side (COR), a sagital section in the vertical direction (SAG) and a transverse section in the vertical direction (TRS). If the moving direction of the invasive device does not change when the position of the invasive device is detected successively, it may be tracked using two orthogonal axis sections. In this case, the imaging sequence is altered to one for imaging two axis sections so as to reduce the imaging time.

When the three-dimensional imaging sequence is employed, the invasive device means can detect the direction of the straight line connecting the distinctive points based on projected images produced by projecting the three-dimensional image onto three planes each including one of the three orthogonal axes. The projection image can be formed by the maximum intensity projection technique (MIP) well known in the art.

As aforementioned, according to the present invention, since the moving direction of an invasive device which changes three-dimensionally can be detected, the tracked object, that is the invasive device, can be tracked without losing sight of it. In addition, since tracking of the invasive device can be performed using the same imaging sequence as that of the imaging scan, real-time navigation excellent in instantaneity can be realized.

Further, the MRI apparatus of the present invention can be provided with navigation means for altering the gradient magnetic field condition of the imaging sequence using the position or the moving direction of the invasive device detected by the invasive device detecting means such that the imaging slice or region includes the invasive device and the region of interest to which the invasive device is guided.

According to this MRI apparatus, the position and moving direction of the invasive device can be detected based on image information obtained by continuously performing imaging of tissues or blood vessels, without need for performing imaging for detecting the position of the invasive device as conventionally required. Therefore, the time lag of detection of the position and moving direction is reduced and the instantaneity of real-time navigation for guiding the invasive device to a region of interest can be improved.

### BRIEF EXPLANATION OF DRAWINGS

FIG. 1 is a block diagram of one embodiment of an MRI apparatus to which the present invention is applied.

FIG. 2 is a block diagram showing elements of the MRI apparatus to which the present invention is applied in detail.

FIG. 3 is a diagram showing the control unit of the MRI apparatus of FIG. 1 in detail.

FIG. 4 is one example of an imaging sequence applicable to the present invention.

FIG. 5 is an explanatory view of a method for defining a mark in the MRI apparatus according to one embodiment of the present invention.

FIG. 6(A) is an example of a monitor image obtained by fluoroscopy according to the embodiment shown in FIG. 5, (B) shows a distance change between a catheter and the center of a region to be observed, and (C) and (D) respectively show a change of image-updating interval according to the present invention and the conventional technique.

FIG. 7 is an explanatory view of narrowing the field of view.

FIG. 8 shows one example of a monitor image obtained by fluoroscopy according to another embodiment of the present invention.

FIG. 9(A) shows a change of the moving speed of a catheter in the embodiment shown in FIG. 8, and (B) and (C) show respectively a change of image-updating interval according to the present invention and the conventional technique.

FIG. 10 shows one embodiment of a catheter used in I-MRI by the MRI apparatus according to the present invention.

FIG. 11 is an explanatory view showing a catheter inserted into a blood vessel.

FIG. 12 is another example of an imaging sequence applicable to the present invention.

FIG. 13 shows three-axis sectional images of a region of interest including a catheter.

FIG. 14 is a three-dimensional image of a region of interest including a catheter.

FIG. 15 shows projection images produced by projecting the three-dimensional image of FIG. 14 in the three orthogonal directions and a synthesized image in which the distinctive points of the catheter are imposed.

FIG. 16 is an explanatory view showing a method of determining the moving direction of the catheter from the projection images of FIG. 15.

### DETAILED DESCRIPTION OF PREFERRED EMBODIMENTS

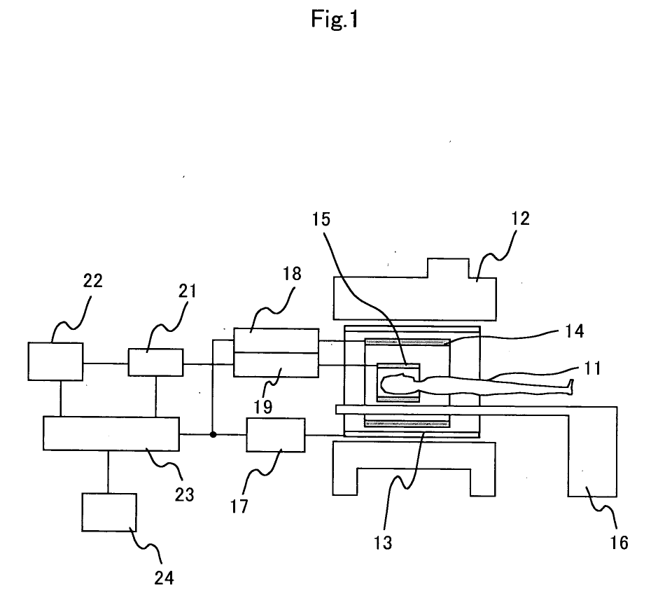

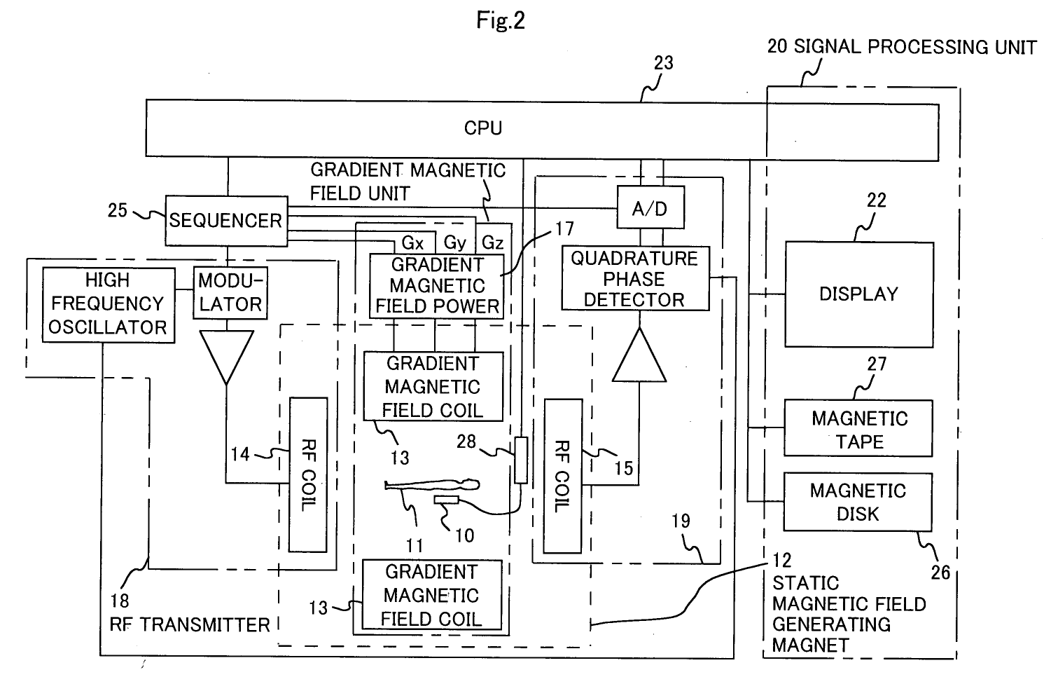

An embodiment of the present invention will hereinafter be explained with reference to FIG. 1-FIG. 6. FIG. 1 shows an overall diagram of a typical MRI apparatus and FIG. 2 shows details of its elements.

As shown in FIG. 1, the MRI apparatus comprises a magnet 12 which generates a static magnetic field in a space (measurement space) where an object to be examined (patient) 11 is placed, a gradient magnetic field generating coil 13 for generating gradient magnetic fields in the measurement space, a high-frequency coil (RF coil) 14 for generating an RF magnetic field in the measurement space, and a high-frequency probe (RF probe) 15 for receiving NMR signals emitted from the object. The patient lying on a bed 16 is inserted into the static magnetic field so that the imaging region is positioned in the measurement space.

The gradient magnetic field coil 13 consists of a plurality of gradient magnetic field coils which generate gradient magnetic fields in the three orthogonal axis directions (X, Y, Z) and is supplied with a pulse-like exciting current by a gradient magnetic field power supply 17 so as to generate a desired gradient magnetic field. The manner of applying the gradient magnetic field can be used to arbitrarily determine the image slice and impart positional information to the NMR signals. The gradient magnetic field coil 13 and the gradient magnetic field power supply 17 constitute a gradient magnetic field unit shown in FIG. 2.

The RF coil 14 generates an RF magnetic field corresponding to an RF magnetic field pulse supplied by an RF transmitter 18. As the transmitting unit, the RF transmitter 18 consists of a high-frequency oscillator, modulator, high-frequency amplifier, and so forth as shown in FIG. 2. The high-frequency pulse output from the high-frequency oscillator is modulated by the modulator, amplified and supplied to the RF coil 14 so that the RF pulse is applied to the object. This RF pulse excites atomic nuclei in tissues of the object to cause nuclear magnetic resonance. Currently common subjects of measurement in clinical settings are spatial distribution of the density of the proton constituting a major part of the patient and spatial distribution of relaxation of the excited state. Images of these spatial distributions facilitate therapies by providing two or three dimensional images of morphology or function of the human head, abdomen, extremities and the like.

NMR signals received by the RF probe 15 are input to a signal detector 19 to be processed, e.g., amplified, detected and the like. The signal detector 19 consists of an amplifier, quadrature phase detector, A/D converter and the like. The NMR signals output from the signal detector 19 are signals processed into image signals by an image reconstructing unit 21. The image signals output from the image reconstructing unit 21 are displayed on a display unit (monitor or display) 22.

The gradient magnetic field power supply 17, RF transmitter 18 and signal detector 19 are controlled by a control unit (CPU) 23 according to a sequence called a pulse sequence. Such control is ordinarily carried out through a sequencer 25 shown in FIG. 2. The control unit 23 also controls the image reconstruction unit 21 and monitor 22, receives image information input from the image reconstruction unit 21 or monitor 22 to perform analysis, and stores required data such as image data in a memory unit (magnetic disc 26, magnetic tape 27 and the like in FIG. 2). An input means is provided for the operator to input various kinds of information into the control unit 23. In FIG. 2, the image reconstruction unit 21, monitor 22 and memory unit are included in a signal processing unit 20.

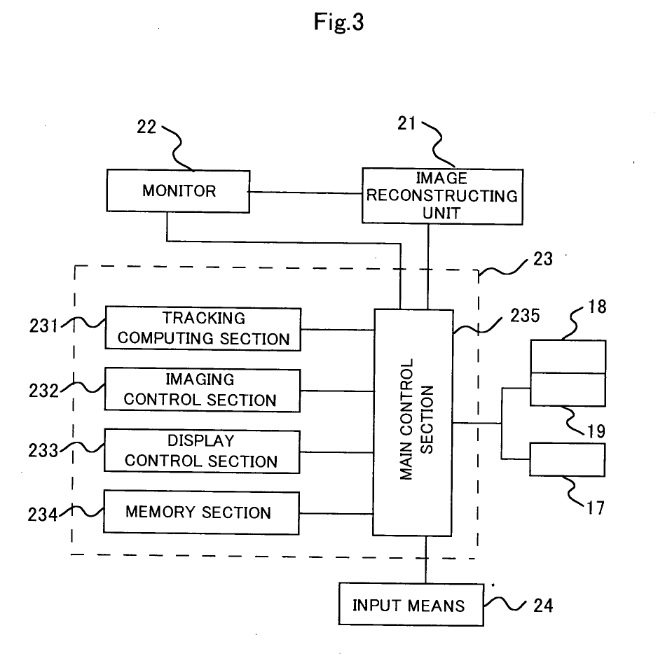

An exemplary configuration of the control unit 23 is shown in FIG. 3. As shown in the figure, the control unit 23 comprises a tracking computing section 231, which calculates the position of an invasive device based on image information from the image reconstruction unit 21 or monitor 22, an imaging control section 232 for controlling the imaging sequence as above-mentioned, a display control section 233 for controlling images displayed on the monitor 22, a memory part 234 for storging imaging parameters and data required for the calculation, and a main control part 235 for integrally controlling all of parts.

The tracking computing section 231 detects the position of the invasive device from the image of the invasive device (distinctive points) displayed as an MR image and the position of marks displayed on the monitor 22 through the input means 24, and calculates the distance between the marked position on the image and the invasive device and the moving speed of the invasive device. The tracking computing section 231 sends instructions to the imaging control section 232 to alter the imaging conditions or imaging sequence based on the calculated distance or speed. The imaging control section 232 controls the gradient magnetic field power supply 17, RF transmitter 18 and signal detector 19 based on the imaging sequence and imaging conditions selected through the input means 24, and also alters the imaging sequence and the imaging conditions based on the position information sent from the tracking computing section 231. The display control section 233 controls the display on the monitor 22.

Next, a method of producing MR images of the object using the thus configured MRI apparatus will be explained. In this embodiment, continuous fluoroscopic imaging is carried out while the invasive device such as a catheter is inserted into the object, and tracking of the invasive device and control of the imaging sequence or imaging conditions are performed in accordance with the position of the invasive device are performed. The invasive device, e.g., a catheter, is equipped with receiving coils or added with a magnetic material so that its position on the image can be distinguished from tissues. Owing to used of the coils or magnetic material, the invasive device is displayed with a different luminance from ordinary regions of the living body so that the tracking computing section 231 can calculate the position thereof.

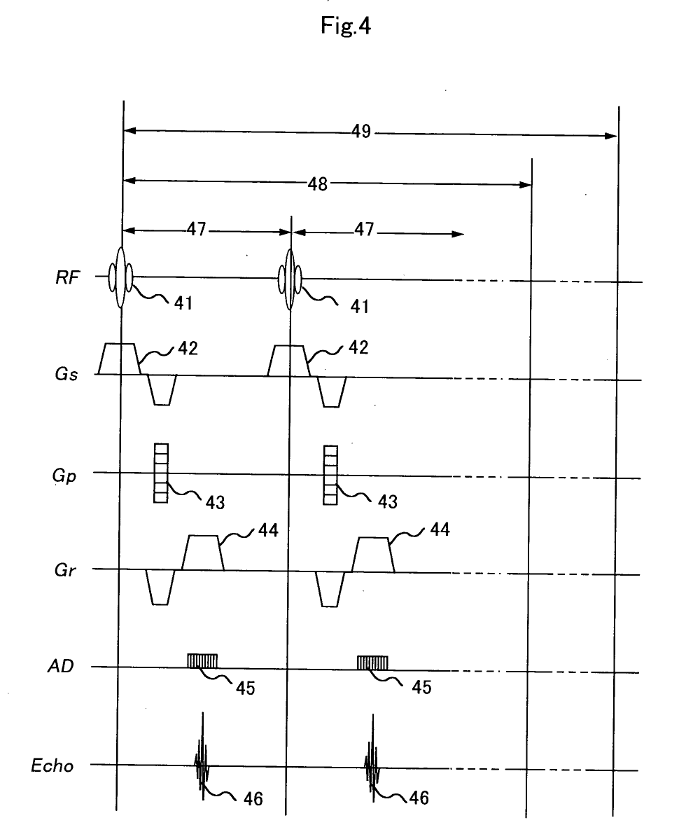

The imaging sequence used for the fluoroscopy may be a gradient echo sequence or a sequence according to echo planar imaging (EPI). FIG. 4 shows the gradient echo sequence as an example of an ordinary imaging sequence. From top to bottom in the figure, RF is a high frequency pulse, Gs is a slice gradient magnetic field, Gp is a phase encoding gradient magnetic field, Gr is a readout gradient magnetic field, AD is a sampling window, and Echo is an NMR signal (echo signal). The ordinate represents intensities and the abscissa represents time.

In this imaging sequence, a slice gradient magnetic field pulse 42 corresponding to a desired slice position is generated and applied to the object together with an RF pulse 41. Protons, for example, within the object are excited by these pulses and an echo signal 46 is emitted from the object. In order to impart the phase information and frequency information, which are spatial position information, a phase encode gradient magnetic field pulse 43 is applied and then a readout gradient magnetic field pulse 44 is applied. During application of the readout gradient magnetic field pulse 44, echo signal 46 is sampled in the sampling window 45.

The above pulse sequence is repeated plural times while changing the intensity of the phase encode gradient magnetic field pulse 43 in order to obtain a two-dimensional image. In FIG. 4, reference symbol 47 indicates the repetition interval of the pulse sequence, reference symbol 48 indicates the image-updating interval of the two-dimensional image, and reference symbol 49 indicates the fluoroscopic imaging time. A phase encode number per image may be selected from among 64, 128, 256, 1024, for example. The echo signal is generally sampled as a time series signals of 128, 256, 512 or 1024 through a sampling window. These echo signals are subjected to the two-dimension Fourier transform to produce a single MR image.

The thus produced MR image is obtained at every repetition time 48 of the pulse sequence. In I-MRI, images continuously obtained during fluoroscopic imaging are displayed on the monitor. Thus, the state of the patient, the position of the invasive device inserted into the patient and the like can be monitored.

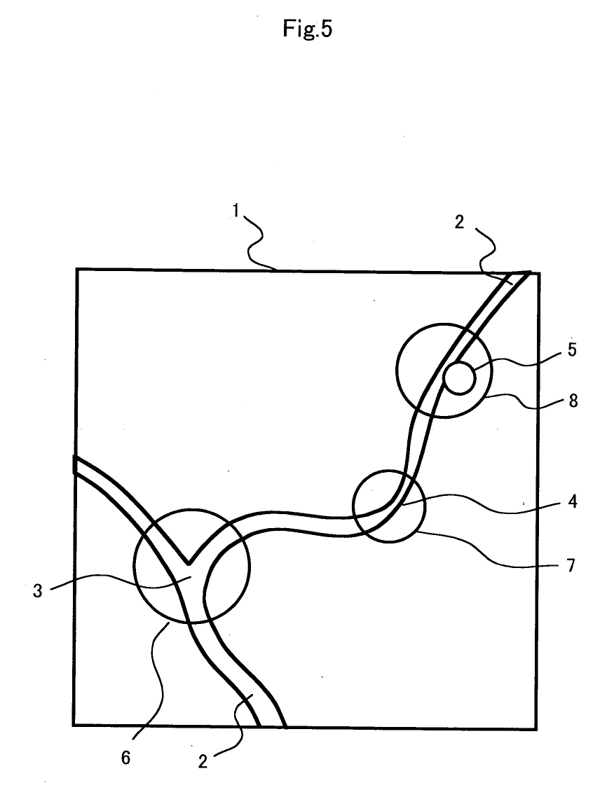

Next, the method of improving the invasive device imaging performance by altering the frame rate or the like in accordance with the position of the invasive device during the fluoroscopic imaging will be explained. FIG. 5 is an explanatory view of one embodiment of the method. The explanation of this embodiment is made regarding the case of inserting a catheter, the invasive device, into a blood vessel to target an aneurysm.

In the embodiment, a tomogram 1 of a desired portion as shown in FIG. 5 is imaged prior to the above-mentioned fluoroscopy. The image 1 displayed on the monitor 22 shows a blood vessel 2, bifurcation 3, constriction 4, and aneurysm 5 that the target of treatment. When a catheter is inserted into a blood vessel, the catheter must generally be operated very carefully at a bifurcation or constriction of the vessel. Accordingly, the operator operates the input means 24 to define circular marks 6, 7 and 8 at the bifurcation 3, constriction 4 and target 5 of the displayed monitor image as regions to be observed (observation regions). The sizes of the marks are changeable between different observation regions.

The shape of the mark is not limited to circular as illustrated in the figure but may instead be a dot or square. When the mark is a dot, it is preferable that a predetermined range centered on the dot be defined as the observation region. When the mark is circular or square, it is preferable to enable the radius of the circle or size of the square to be defined arbitrarily.

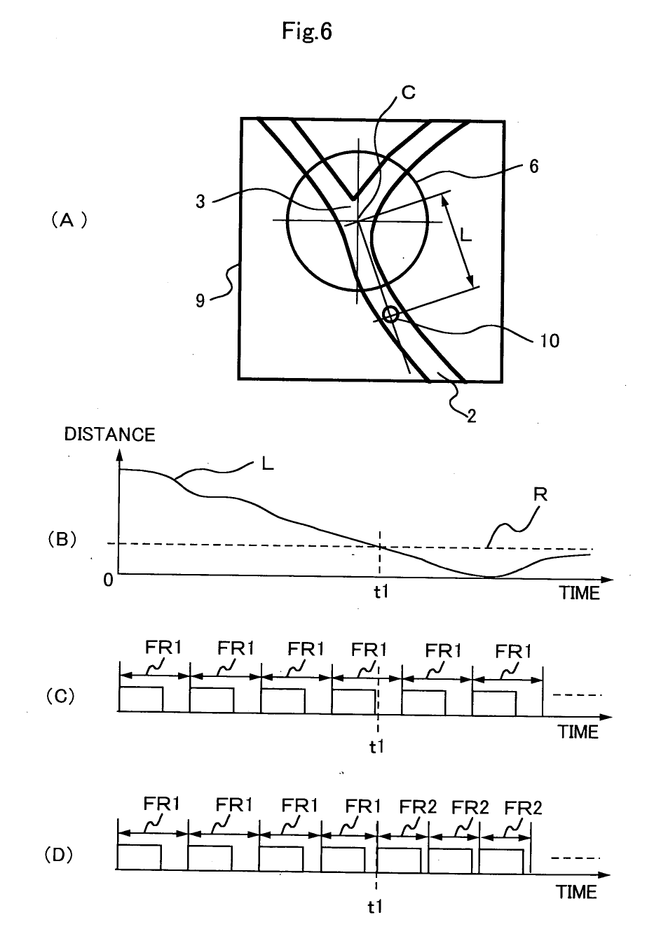

After the observation region is defined in the aforementioned manner, fluoroscopic imaging is started while inserting the catheter. Thus, a continuously obtained time-series image is displayed on the monitor 22. An example of the time-series image 8 is shown in FIG. 6(A). As shown in the figure, the mark 6 of the observation region and the catheter 10 are displayed on the monitor. The control unit 23 (tracking computing section 231) successively detects the position of the catheter in the image from the continuously obtained time-series image. As mentioned previously, since the magnetic material is admixed in the catheter 10, it is displayed with a different luminance from ordinary region of the object and can be easily detected by the control unit 23. Specifically, for example, a set of pixels having a luminance equal to or higher than a predetermined threshold value is extracted and the ordinate of the center or gravity center of the set is determined to ascertain the center position of the tip of the catheter. Alternatively, a position at a predetermined distance from the thus found ordinate is determined as the position of the tip of the catheter. The position of the catheter 10 is detected from the predetermined ordinate of the image. Then, the control unit 23 successively determines the straight-line distance L between the center C of the mark 6 and the center of the catheter 10 by calculation.

FIG. 6(B) shows an example where the position of the catheter 10 is thus tracked and the distance L from the near mark 6 is found. As shown in the figure, the catheter 10 approaches the mark 6 as time passes and, at time t1, enters within the radius R of the mark 6. As further time passes, the catheter 10 goes through the center of the mark 6 and departs from it. FIGS. 6(C) and (D) show the image-updating interval FR in the fluoroscopy. FIG. 6(C) relates to conventional fluoroscopy in which imaging is performed with a fixed image-updating interval RF1. FIG. 6(D) shows that the image-updating interval is altered to FR2, which is shorter than FR1, in response to the positional relationship between the catheter 10 and mark 6. Since careful operation is required near the observation region, the control unit 23 (imaging control section 232) changes the image-updating interval to make it shorter as the catheter approaches the center of mark 6.

Altering of the image-updating interval is determined, for example, by calculation using a function defining the relation between the image-updating interval and the distance L, which is defined beforehand. Alternatively, a table in which the image-updating interval is defined relative to distance L is prepared beforehand, when the mark of the observation region is defined. Then, the image-updating interval is changed based on the table. The imaging sequence may be altered in a different manner for each observation region, i.e., mark. For example, the image-updating interval may be altered in accordance with the size of the radius R of the observation region. Further, the image-updating interval may be directly input and defined when the mark is defined on the image. Further, a degree of interest may be defined for each mark when the mark is defined. Specifically, plural stages, e.g., three stages, of the image-updating interval may be defined in association with the degree of interest and the distance L.

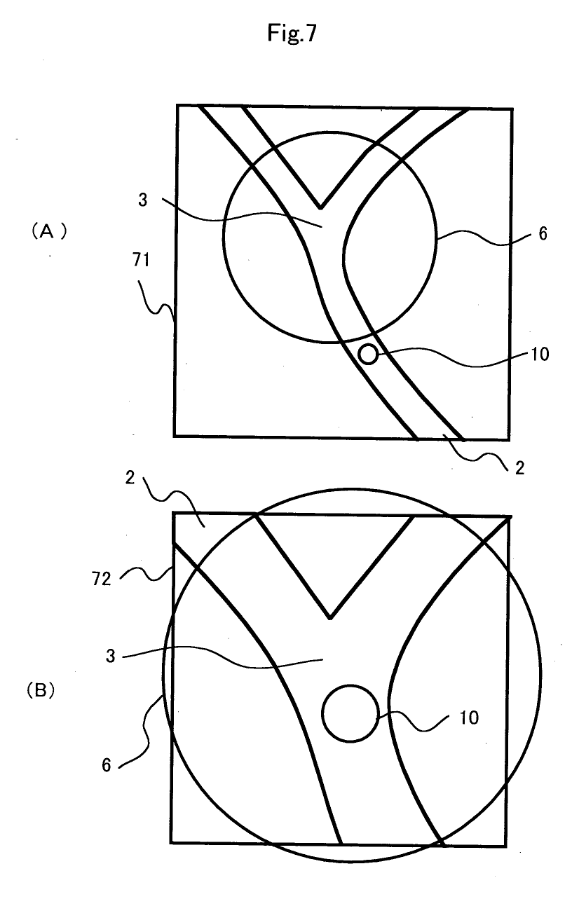

Another example of altering the imaging condition in accordance with the distance L, i.e., altering of the spatial resolution is shown in FIG. 7. In this case, the method of tracking the position of the catheter 10 and the method of determining the distance from the nearest mark 6 are the same as in the aforementioned example. However, in FIG. 7, when the catheter 10 goes into the mark, the spatial resolution is improved by making the imaging field of view small so as to display an enlarged observation region. Specifically, when the catheter 10 is outside the mark 6 region, an imaging sequence for a large field of view is performed so that the image 71 as illustrated in FIG. 7(A) is displayed. When the catheter 10 goes into the mark 6, the imaging sequence is altered to that for a small field of view so that the image 72 as illustrated in FIG. 7(B) is displayed. By this alteration, the operator can carry out the insertion operation while observing the movement of the catheter 10 finely. In this case also, the value of the spatial resolution can be altered corresponding to the degree of interest.

Alteration of the spatial resolution is not limited to that performed by making the field of view small but can also be performed by changing the imaging sequence. Specifically, the phase encoding number and the sampling number of echo signals may be changed so as to improve the spatial resolution.

According to the embodiment shown in FIGS. 5-7, since the imaging speed automatically increases or the spatial resolution becomes high when the catheter 10 reaches the marked range of the observation region, the operator can finely monitor the movement of the catheter 10 and the positional relationship with the blood vessel in the image with high accuracy.

When imaging a blood vessel to monitor an invasive device such as a catheter, it is preferable to enhance the contrast of the blood vessel by injecting a contrast agent into the vessel. When the present invention is combined with this contrasting technique, the injection timing of the contrast agent can be determined properly by, for example, automatically starting the injection of the contrast agent at the time when the device goes into the marked area.

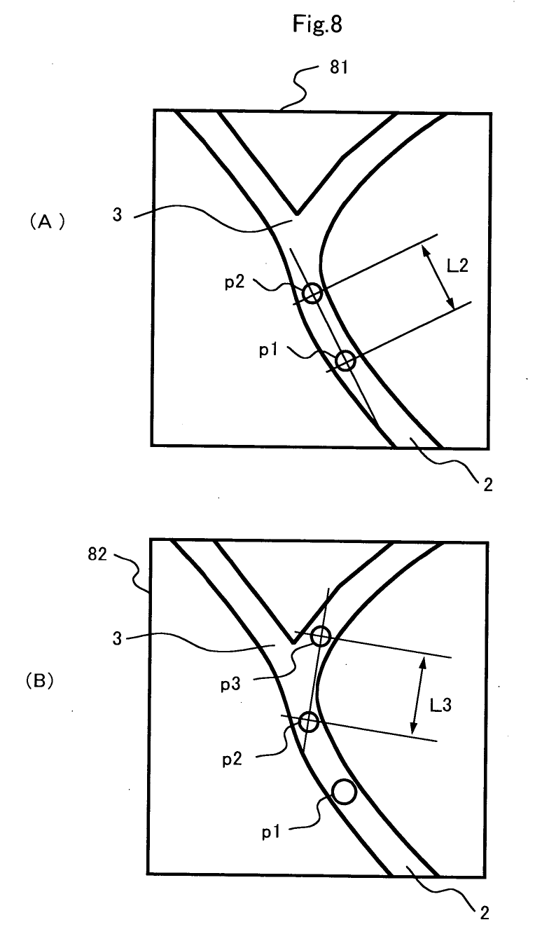

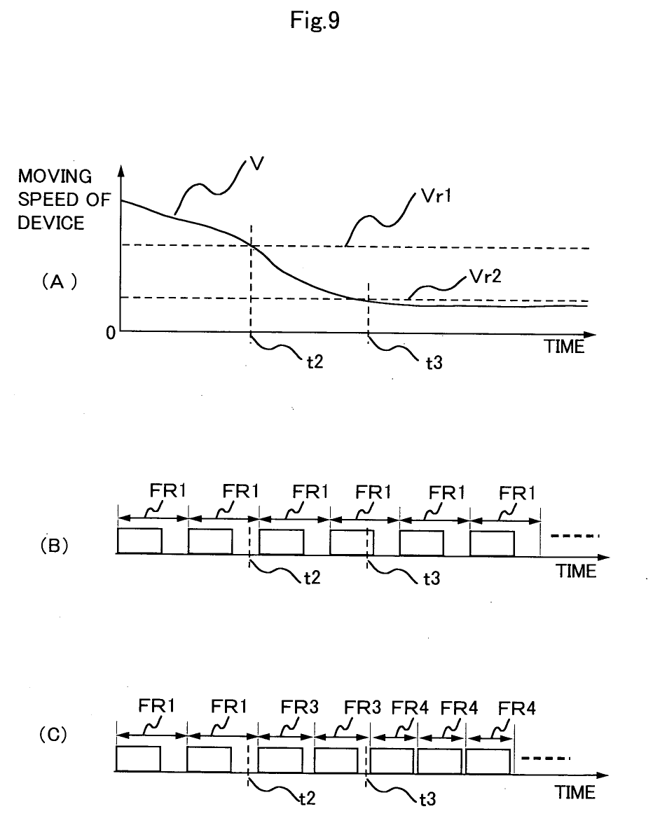

Next, another embodiment will be explained with reference to FIGS. 8 and 9. In this embodiment, the image-updating interval FR or the spatial resolution of the time-series image is altered in accordance with the moving speed of the invasive device. The moving speed of the invasive device can be calculated from two images obtained at different imaging times. FIG. 8(A) illustrates an image 81 in which positions P1 and P2 of the catheter 10, which were detected from two images obtained at different imaging times by fluoroscopy, are indicated simultaneously. The figure shows that the catheter 10 moved from P1 to P2 owing to insertion operation. The average moving speed V1 of the catheter 10 here is found by dividing the distance L2 between the positions P1 and P2 by the imaging time difference ΔT1 between the two images. That is,

V1=L2/ΔT1

With further passage of time, the catheter 10 proceeds from position P2 by distance L3 to position P3 shown in FIG. 8(B). Where the imaging time difference between the two images is ΔT2, then the average moving speed V2 of the catheter 10 here is found by the following equation:

V2=L3/ΔT2

Such a calculation of the speed may be done by, for example, the tracking computing section 231.

As mentioned previously, since the operator instinctively performs insertion operation carefully in the neighborhood of the observation region, the moving speed of the catheter decreases. Accordingly, when the insertion speed of the catheter is slow, the image-updating interval is shortened or the spatial resolution is improved so as to improve the catheter imaging ability.

FIG. 9 shows an example where the image-updating interval FR is altered in accordance with the average moving speed of the catheter 10. FIG. 9(A) illustrates change of the moving speed V of the catheter 10. The moving speed V is compared with two threshold values Vr1 and Vr2. If Vr2<V≦Vr1, the image-updating interval is set to FR3 and if the V=Vr2, the image-updating interval is changed to FR4. The moving speed V of the catheter 10 is slowed down to the threshold value Vr1 or less at t2 and further to the threshold value Vr2 or less at t2. FIG. 9(B) shows that monitoring is carried out with a constant image-updating interval FR1 during fluoroscopic imaging as in the conventional imaging. FIG. 9(C) shows the image-updating interval changed from FR1 to FR3 and to FR4 (FR1→FR3→FR4) in accordance with the moving speed of the catheter by the method of the present embodiment.

This embodiment provides the same effect as that obtained by the embodiment shown in FIGS. 5-7, and yet is advantageous since of the need to mark the observation region is obviated. In this embodiment too, the spatial resolution of images can be altered in accordance with the moving speed of the invasive device, alone or in combination with the image-updating interval. In order to improve the spatial resolution of the image, the pulse sequence may be altered so as to make the field of view small or so as to increase the phase encode number and sampling number.

While the MRI apparatus according to one aspect of the present invention and IMR using the MIR apparatus have been explained hitherto, the present invention is not limited to the aforementioned embodiments and various other embodiments are also possible within the scope of the invention. For example, while the gradient echo method is employed as an imaging sequence for fluoroscopy in the aforementioned embodiments, echo planar imaging (EPI), one of the fast imaging methods, can be employed. The echo-sharing method mentioned previously can also be used in combination.

Next, the MRI apparatus according to another aspect of the present invention will be explained. The configuration of this MRI apparatus is similar to that of the MRI apparatus shown in FIGS. 1-3. However, it is characterized in having as its invasive device tracking capability (a tracking computing section) the capability to detect three-dimensional position and direction. Preferably, it has navigation capability for guiding the operator's insertion operation by displaying the tracking results of the invasive device on the monitor 22. The tracking capability and navigation capability are realized as functions of the tracking computing section 231 and imaging control section 232 of the control unit 23.

In addition, in order to realize the tracking capability, an invasive device equipped with at least two distinctive points spaced in the longitudinal direction is used. Each distinctive point is defined as a point which is distinguishable from the other parts (for example, having higher luminance) in the MRI image, and produced by installing a receiving coil or adding a magnetic material.

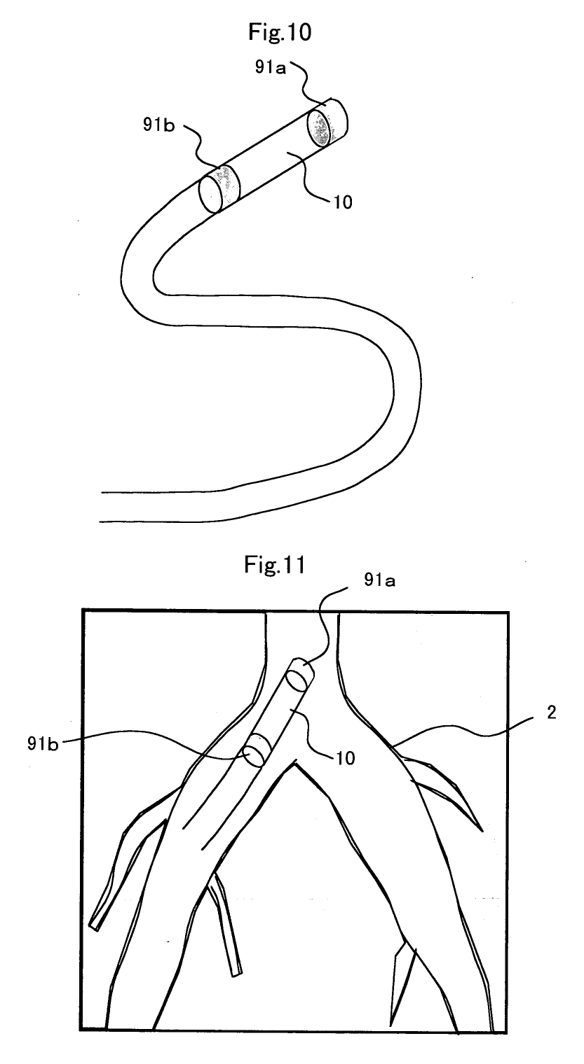

The embodiments will be explained in detail with reference to FIGS. 10-14 hereinafter.

FIG. 10 illustrates a catheter 10 as an example of the invasive device. The catheter 10 has a cylindrical shape capable of being inserted into blood vessels and is provided with two receiving coils 91a and 91b embedded in the tip at an interval. The interval between the receiving coils 91a and 91b is typically 3-5 cm. The echo signals received by the receiving coils 91a and 91b are transmitted to the signal detector (receiving unit 28 in FIG. 2) through a signal line (not illustrated). The receiving coils embedded in the catheter may be loop coils or linear coils.

The receiving unit 28 has the same configuration as the receiving unit 19 shown in FIG. 2 for receiving signals from the ordinary receiving coil 25. The signal processing unit 20 produces synthesized images using the signals from these two systems if necessary.

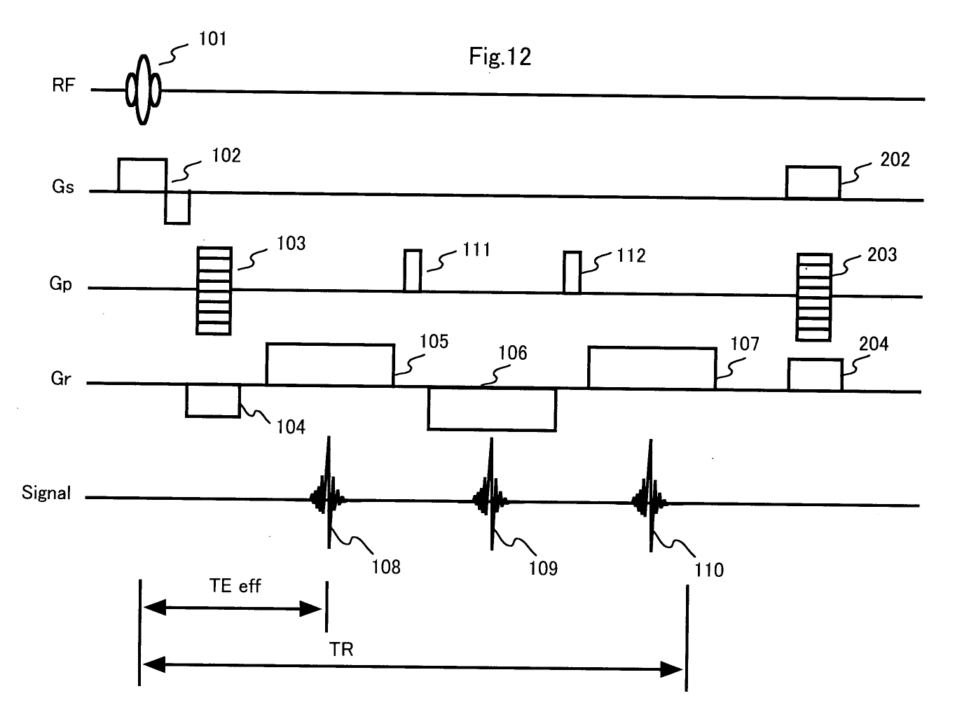

Upon imaging, the patient 11 is placed within a measurement space in a static magnetic field of the MRI apparatus and, as shown in FIG. 11, the catheter is inserted through the blood vessel 2 to a region to be treated. In the process of insertion, continuous imaging is performed to obtain MR images. As the imaging sequence used for the continuous imaging, the sequence of the gradient echo method as shown in FIG. 3 may be applied but an imaging sequence according to a multi-shot EPI will be explained as another example.

FIG. 12 shows the two-dimensional imaging sequence according to the multi-shot EPI method. From top to bottom in the figure are illustrated a high-frequency pulse RF, slice gradient magnetic field Gs, phase encode gradient magnetic field Gp, readout gradient magnetic field Gr and echo signal. The abscissa represents time and the ordinate represents intensities.

As illustrated, first, a high-frequency pulse RF 101 is applied together with a slice gradient magnetic field 102 to excite an imaging slice of the object in this imaging sequence. The high-frequency pulse 101 has a flip angle α° of 90° or less. Then, after a phase encode gradient magnetic field 103 and magnetic field pulse 104 in the readout direction are applied, readout magnetic field pulse 105, 106 and 107 are applied while the polarity is reversed repeatedly to acquire a plurality of echo signals 108, 109 and 110. Between the readout gradient magnetic field pulses 105 and 106, and between the readout gradient magnetic field pulses 106 and 107, blip-like gradient magnetic fields 111 and 112 are applied in the phase encode direction to make the phase of each echo signal different.

Such an imaging sequence is repeated with a repetition time of TR to acquire echo signals necessary for reconstructing one image. In FIG. 12, gradient magnetic fields 202, 203 and 204 are applied in the end of the repetition in order to eliminate effect of each gradient magnetic field applied within the repetition. Such an imaging sequence of multi-shot EPI is repeatedly performed and the set of echo signals thus measured is subjected to an image reconstruction operation such as Fourier transform to produce a two-dimensional image. With TR of 10 ms, echo interval TE of 4 ms, FOV (field of view) of 260, number of data in the readout direction of 128, phase encode amount of 120, shot number of 40 and number of echo trains of 3, a single two-dimensional image can be continuously obtained with an updating interval of about 0.4 seconds (10*120/3=400 ms).

Next, a method of detecting the position and moving direction of the catheter 10 based on the thus continuously obtained MR images will be explained. If the catheter is in the imaging area, a two-dimensional image as shown in FIG. 11 is displayed but the direction of the catheter cannot be detected from the two-dimensional image. Accordingly, in the present embodiment, three-axis sections, for example, three sections respectively including an X-axis, Y-axis and Z-axis are imaged as slice axes. With the aforementioned parameters, the time required for imaging is 1.2 seconds (0.4 seconds*3 slices).

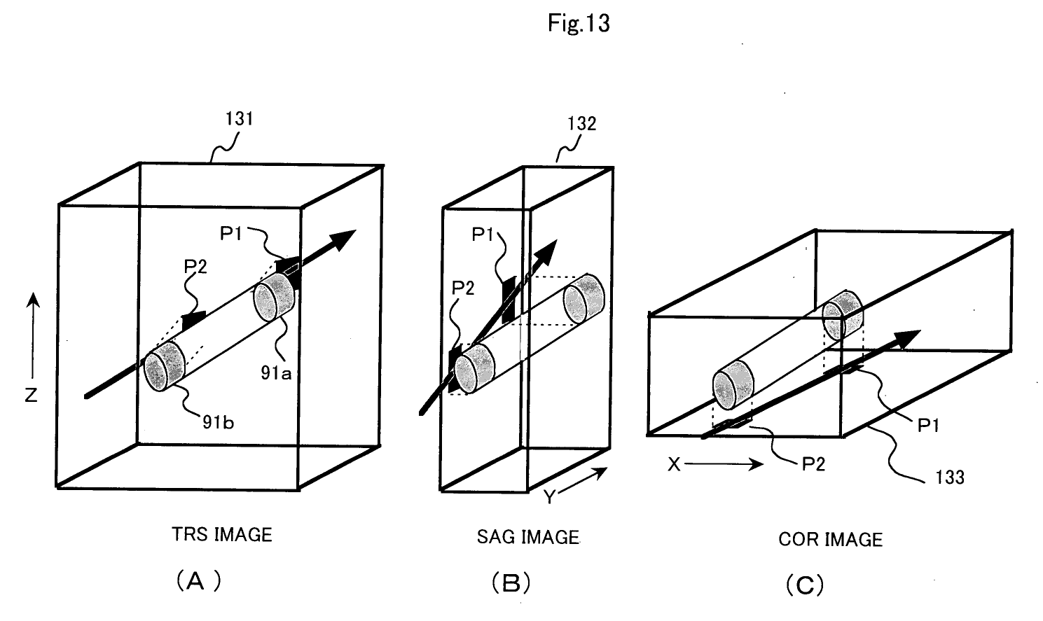

Conceptual views of the thus obtained images are illustrated in perspective in FIG. 13. In FIG. 13, (A) is a TRS image 131, i.e., a tomogram along with the Z-axis, (B) is an SAG image 132, i.e., a tomogram along with the Y-axis, and (C) is a COR image 133, i.e., a tomogram along with the X-axis. These drawings are represented taking account of the slice thickness of the imaging region. The thickness is typically 100 mm or less and is preferably made thinner (for example 10 mm or less) as the catheter approaches the region of interest.

In the images 131-133, portions corresponding to the receiving coils 91a and 91b are indicated as images having markedly different luminance from the other parts as distinctive points P1 and P2 of the image. The control unit 23 (CPU) detects the position and direction of the catheter based on the three-axis tomograms. The method of detecting the positions P1 and P2 of the catheter is similar to that in the aforementioned embodiment. The moving direction is determined by, for example, geometrically calculating the angles θx, θy and θz that a straight line connecting the distinctive points P1 and the distinctive points P2 makes with each axis X, Y and Z in each image and synthesizing the angles to determine the three-dimensional direction. For finding the three-dimensional direction, it is convenient to use the conventional polar coordinate system or cylindrical coordinate system. The overall moving directions of the catheter, which are shown by arrows in the drawings, are detected by comparison with the previously obtained image of time-series data.

The three-axis tomograms of a two-dimensional I-MRI image are thus obtained using the same imaging sequence as that of the imaging scan, and tracking is performed by detecting the three-dimensional position and moving direction of the catheter from these images.

According to this embodiment, it is not necessary to perform the imaging scan and the scan for detecting the catheter separately, and yet detection of the position and direction of the catheter can be done in a short time (within 1.2 seconds, for example) so that navigation in real time can be realized. Specifically, when the imaging conditions such as slice position and slice direction are automatically altered in accordance with the detected position and direction of the catheter, the catheter can be guided to the region of interest on the image without being lost sight of.

Further, since the three-axis tomograms are used for detecting the position and direction of the catheter in this embodiment, even if the invasive device is detected by only one of the tomograms, the imaging region can be corrected in the following imaging sequence referring to the positional information and can be followed well. That is, complete disappearance of the invasive device can be avoided and, if the approximate position can be ascertained, the exact position can be grasped from the next obtained image.

If the moving direction does not change greatly, the catheter is not likely to go out of sight even if the three-axis tomograms are not obtained. In this case, detection of the position and direction may be performed based on the two-axis tomograms to reduce the detecting time. This enables a further improvement in the instantaneity (real time property) of the detection.

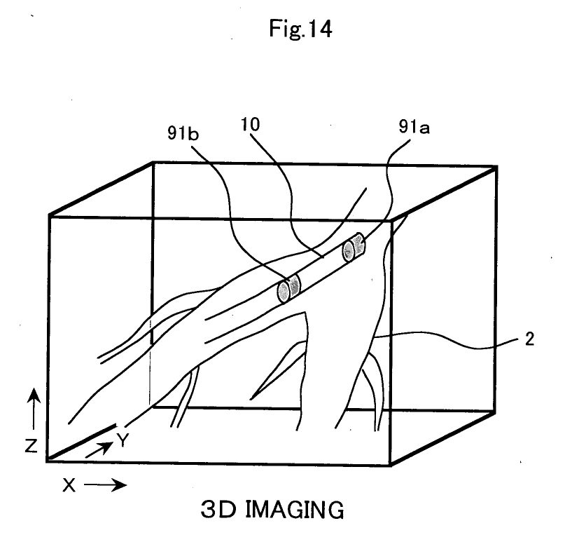

Another embodiment of the present invention, in which the position and moving direction of the catheter 10 are detected while performing three-dimensional I-MRI imaging, will be explained with reference to FIGS. 14-16. In this case too, the multi-shot EPI sequence as shown in FIG. 12 or the sequence according to the gradient echo method can be employed as the imaging sequence but an encoding loop in the slice direction is included in addition to the phase encode loop (repetition while changing the phase encode amount) shown in, for example, FIG. 12. Such a three-dimensional imaging also produces a three-dimensional image with a 1.2 second interval with the slice encode number of 3 and the same parameters as those in the aforementioned example for the other conditions. However, the slice encode number is not limited to 3 and can be increased to the extent that it does not degrade instantaneity.

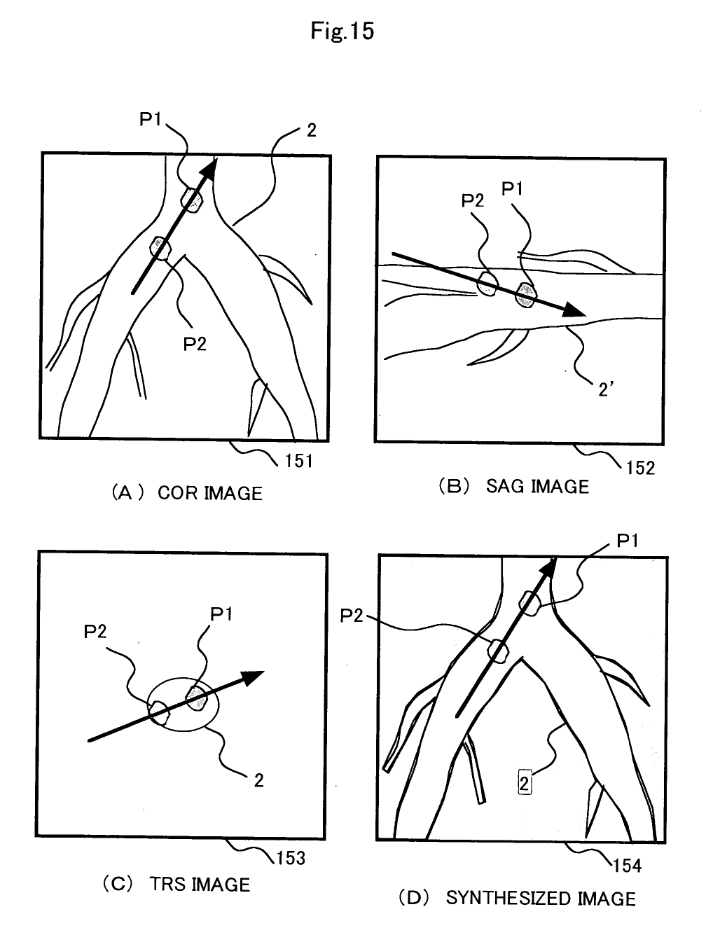

An example of a three-dimensional image of the region of interest including the catheter, which is obtained by performing such a three-dimensional imaging sequence, is shown in FIG. 14. FIG. 14 is an illustrative view of the catheter 10 inserted into the blood vessel 2. The control unit 23 detects the position and moving direction of the catheter based on the data of such a three-dimensional image. Therefore, the three-dimensional image data is projected in the three-axis directions and processed with a maximum intensity projection (MIP) which produces an image using the pixels having the maximum value. Thus, as shown in FIGS. 15(A), (B) and (C), a COR image 151, SAG image 152 and TRS image 153 are obtained. FIG. 15(D) is an exemplary view of a synthesized image produced by superimposing the detected distinctive points P1 and P2 of the catheter on a tomogram including the region of interest obtained prior to insertion of the catheter. These images are MPR (Multi Planar Projection) images.

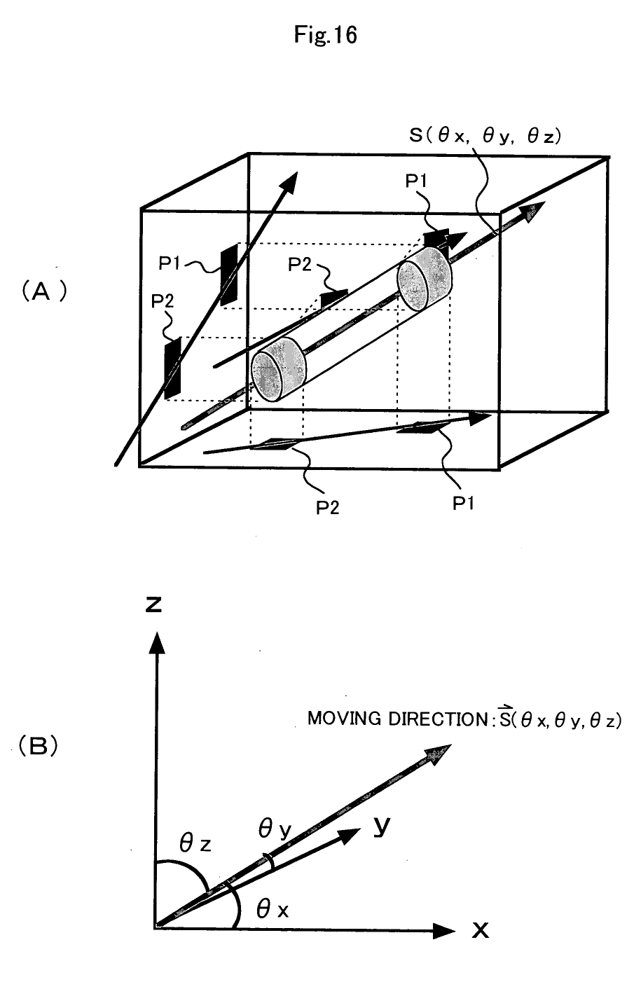

Next, the control unit 23 detects the position and moving direction of the catheter based on the COR image 151, SAG image 152 and TRS image 153 shown in FIG. 15. The method of detection is similar to detection from the three-axis tomograms produced by the two-dimensional imaging. For example, as shown in FIGS. 16(A) and (B), the angles θx, θy and θz that a straight line connecting the distinctive point P1 and the distinctive point P2 make with each axis X, Y and Z in each image are calculated geometrically and synthesized to determine the three-dimensional direction S(θx, θy, θz). Although an orthogonal coordinate system is used to simplify the explanation, it is convenient to use the well-known polar coordinate system when the three-dimensional moving direction is determined.

In this embodiment too, detection of the position and moving direction of the catheter can be achieved using the same imaging sequence as that of the three-dimensional I-MRI imaging. Therefore, a MIP image, position and moving direction of the catheter can be continuously acquired and displayed in a short time (within 1.2 seconds when the slice encode number is 3). Thus navigation can be realized in real time. The distance that the catheter moves and the moving speed may be also determined and displayed if necessary.

While detection of the position and moving direction of the catheter has been mainly explained hitherto, the MRI apparatus of the present invention may be also provided with a navigation means for performing imaging while tracking the catheter by automatically altering the imaging position in response to the operator's instructions. Such a means can be realized as a function of the imaging control section 232 of the control unit 23 shown in FIG. 3. The imaging control section 232 alters the gradient magnetic field conditions of the imaging sequence using the detected position and moving direction so that the imaging slice or region includes both the region of interest and the catheter.

The navigation capability realized in such a manner does not require imaging for detecting the catheter position beyond that of the imaging scan, and while imaging of the tissue is carried out continuously, the position and moving direction of the catheter are detected successively using the tissue image information. Accordingly, the imaging time and image processing time can be shortened and the instantaneity (the real time property) of the navigation for guiding the catheter to the portion of interest can be improved. In this case, if tissues surrounding the portion of interest are imaged and stored prior to treatment and the tracking image of the catheter is superimposed and displayed on the tissue images, then the navigation of guiding the catheter to the portion of interest can be done in real time and the visibility of the I-MR image can be further improved.

While the second aspect of the present invention has been explained hitherto referring to embodiments, the present invention is not limited to the aforementioned embodiments and can be modified in various manners. For instance, while the imaging conditions such as slice position and slice direction are automatically altered in accordance with the position and moving direction of the catheter detected by the control unit 23 in the aforementioned embodiments, the operator may set parameters of the imaging sequence such as TR/TE, slice direction, slab number, and the like to the control unit 23 through the input means 24. In this case, it is possible, for example, to display the navigation image of the desired slice direction or contrast during I-MRI in accordance with the measured portion or the circumstances.

Further, while receiving coils were exemplified as the indicators for distinctive points of the catheter in the aforementioned embodiment, the catheter may be equipped with markers made of a low signal material such as a magnetic material or a high signal material instead of the receiving coils, or such a material may be included in a resin constituting the catheter. The invasive device is not limited to the catheter, as exemplified, and the present invention can be applied for tracking and navigation of a biopsy needle or any other device inserted into an object.

Further, while the MRI apparatus according to the first aspect of the present invention and the MRI apparatus according to the second aspect have been explained separately, the MRI apparatus may be provide with the first and second aspects together. Specifically, mainly as a function of the control unit of the MRI apparatus, the MRI apparatus may be made capable of altering the temporal resolution or spatial resolution of MR images in accordance with the position of the invasive device together with of the capability of tracking the three-dimensional position and moving direction of the invasive device based on MR images obtained as three-axis tomograms or three-dimensional image.

What is claimed is:

1. A magnetic resonance imaging apparatus comprising: control means for performing an imaging sequence for measuring nuclear magnetic resonance signals generated by exciting an object to be examined while imparting a spatial position information to the signals; image construction means for producing magnetic resonance images of the object based on the nuclear magnetic resonance signals; display means for displaying the images produced by said image construction means; and input means for defining marks at arbitrary positions of the image displayed on the display means, wherein said control means has a function of altering the imaging sequence when the distance between the invasive device and the mark in the displayed image is within a predetermined range.

2. A magnetic resonance imaging apparatus comprising: control means for performing an imaging sequence for measuring nuclear magnetic resonance signals generated by exciting an object to be examined while imparting a spatial position information to the signals; image construction means for producing magnetic resonance images of the object based on the nuclear magnetic resonance signals; display means for displaying the images produced by the image reconstruction means; and input means for defining marks at arbitrary positions of the image displayed on said display means, wherein said control means has a function of altering at least one of the frame rate and the spatial resolution of the image when the distance between the invasive device and the mark in the displayed image is within a predetermined range.

3. The magnetic resonance imaging apparatus of claim 2, wherein said control means alters at least one of the frame rate and the spatial resolution of the image by altering the imaging sequence.

4. The magnetic resonance imaging apparatus of claim 2 or claim 3, wherein said control means shortens the frame rate of the image as the distance between the invasive device and the mark in the displayed image decreases.

5. The magnetic resonance imaging apparatus of any one of claims 2-4, wherein said control means controls altering of the frame rate based on a predetermined relationship between the frame rate and a distance between the invasive device and mark.

6. The magnetic resonance imaging apparatus of claim 2 or claim 3, wherein said control means alters the spatial resolution to a higher value when the distance between the invasive device and the mark in the displayed image is within a predetermined range.

7. The magnetic resonance imaging apparatus of claim 6, wherein said control means alters the spatial resolution to a higher value by narrowing the image field of view.

8. The magnetic resonance imaging apparatus of claim 6, wherein said control means alters the spatial resolution to a higher value by increasing the phase encode number and echo-signal sampling number of the imaging sequence.

9. The magnetic resonance imaging apparatus of clam 1 or claim 2, wherein said control means has a tracking function for tracking the invasive device by detecting a position of the invasive device in the image.

10. The magnetic resonance imaging apparatus of clam 9, wherein said control means detects the position of the invasive device in the image based on the coordinates of pixels having a luminance equal to or more than a threshold value.

11. The magnetic resonance imaging apparatus of clam 1 or claim 2, wherein the mark is a point indicating an arbitrary position or circle or square enclosing the arbitrary position.

12. The magnetic resonance imaging apparatus of any one of clam 1, claim 2 and claim 11, wherein a plurality of marks can be defined.

13. The magnetic resonance imaging apparatus of claim 12, wherein the size of the mark is changeable for each position.

14. A magnetic resonance imaging apparatus comprising: control means for performing an imaging sequence for measuring nuclear magnetic resonance signals generated by exciting an object to be examined while imparting a spatial position information to the signals; image construction means for producing magnetic resonance images of the object based on the nuclear magnetic resonance signals; and display means for displaying the images produced by said image construction means, wherein said control means has functions of detecting the invasive device in the image, calculating a moving speed of the invasive device and altering the imaging sequence in accordance with the calculated moving speed of the invasive device.

15. A magnetic resonance imaging apparatus comprising: control means for performing an imaging sequence for measuring nuclear magnetic resonance signals generated by exciting an object to be examined while imparting a spatial position information to the signals; image construction means for producing magnetic resonance images of the object based on the nuclear magnetic resonance signals; and display means for displaying the images produced by said image construction means, wherein said control means has functions of detecting the invasive device in the image, calculating a moving speed of the invasive device and altering at least one of the frame rate or the spatial resolution of the image in accordance with the calculated moving speed of the invasive device.

16. The magnetic resonance imaging apparatus of claim 14, wherein said control means alters at least one of the frame rate and spatial resolution of the image by altering the imaging sequence.

17. The magnetic resonance imaging apparatus of claim 14 or claim 15, wherein said control means calculates the moving speed of the invasive device from positions of the invasive device in at least two images obtained at different times.

18. The magnetic resonance imaging apparatus of claim 15 or claim 16, wherein said control means increases the frame rate of the image or the spatial resolution when the moving speed of the invasive device is less than a determined value.

19. The magnetic resonance imaging apparatus of claim 15 or claim 16, wherein said control means controls altering of the frame rate based on a predetermined relationship between the frame rate and the moving speed.

20. The magnetic resonance imaging apparatus of claim 18, wherein said control means alters the spatial resolution to a higher value by narrowing the image field of view.

21. The magnetic resonance imaging apparatus of claim 18, wherein said control means alters the spatial resolution to a higher value by increasing the phase encode number and the sampling number of echo signals of the imaging sequence.

22. A magnetic resonance imaging apparatus comprising: control means for repeatedly performing an imaging sequence for measuring nuclear magnetic resonance signals generated by exciting an object to be examined while imparting a spatial position information to the signals; and image construction means for continuously producing and displaying magnetic resonance images of the object based on the nuclear magnetic resonance signals, wherein said control means comprises invasive device detecting means for detecting distinctive points incorporated in an invasive device inserted into the object for producing at least two distinctive image data based on the magnetic resonance image and detecting a position and three-dimensional moving direction of the invasive device by determining the direction of a line connecting the two distinctive points.

23. The magnetic resonance imaging apparatus of claim 22, wherein said invasive device detecting means detects the three-dimensional moving direction of the invasive device from angles between a line connecting the two distinctive points and three orthogonal axes.

24. The magnetic resonance imaging apparatus of claim 22 or claim 23, wherein the distinctive points are made of small receiving coils.

25. The magnetic resonance imaging apparatus of claim 24, wherein said image construction means comprises two signal processing systems including a signal processing system for processing signals from the small receiving coils.

26. The magnetic resonance imaging apparatus of claim 22, wherein the imaging sequence includes an imaging sequence for imaging tomogram images of three orthogonal axes and said invasive device detecting means determines the direction of a line connecting the distinctive points from the tomogram images of three orthogonal axes.

27. The magnetic resonance imaging apparatus of claim 22, wherein the imaging sequence includes an imaging sequence for three-dimensional imaging and said invasive device detecting means determines the direction of a line connecting the distinctive points from projection images produced by projecting the three-dimensional image in planes including the three axes.

28. The magnetic resonance imaging apparatus of any one of claims 22-27, which apparatus is provided with navigation means for altering the gradient magnetic field condition of the imaging sequence using the position or moving direction of the invasive device detected by said invasive device detecting means such that the imaging slice or imaging region includes both the region of interest to which the invasive device is to be guided and the invasive device.

29. The magnetic resonance imaging apparatus of any one of claims 22-28, wherein said control means alters the thickness of slices in the imaging sequence as the invasive device proceeds.

30. The magnetic resonance imaging apparatus of claims 28, wherein said control means comprises input means for changeably setting parameters of the imaging sequence.

31. The magnetic resonance imaging apparatus of any one of claims 22-30, which apparatus has a function of altering at least one of the frame rate and spatial resolution of the magnetic resonance image in accordance with a position of the invasive device.
